(19) United States
(12) Patent Application Publication
(10) Pub. No.: US 2014/0227205 A1
(43) Pub. Date: Aug. 14, 2014

(54) TOPICAL COMPOSITION

(71) Applicant: Christine Mendrok-Edinger, Basel (CH)

(72) Inventor: Christine Mendrok-Edinger, Basel (CH)

(73) Assignee: DSM IP ASSETS B.V., Heerlen (NL)

(21) Appl. No.: 14/124,762

(22) PCT Filed: Jun. 6, 2012
(86) PCT No.: PCT/EP2012/060676

§ 371 Date: Mar. 28, 2014

(30) Foreign Application Priority Data
Jun. 8, 2011 (EP) 11004658.8
Jun. 15, 2011 (EP) 11169927.8
Jun. 15, 2011 (EP) 11169934.3
Jul. 12, 2011 (EP) 11173617.9

(51) Int. Cl. A61K 8/49; A61Q 17/04; A61K 8/41

(52) U.S. Cl. CPC A61K 8/496; A61K 8/4966; A61K 8/415; A61Q 17/04; A61K 2800/10
USPC 424/59

(57) ABSTRACT

The present invention relates to topical compositions comprising at least one benzotriazol derivative and at least one UV filter substance selected from the group consisting of a triazine derivative and an amino substituted hydroxybenzophenone as well as mixtures thereof.

The present invention relates to topical compositions comprising at least one benzotriazol derivative and at least one UV filter substance selected from the group consisting of a triazine derivative and an amino substituted hydroxybenzophenone as well as mixtures thereof.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancer. Thus, today's focus is towards eliminating as much of UVA (320-400 nm) and/or UVB (280-320 nm) light as possible. Consequently, there's a constantly increasing need for sun care products exhibiting high SPF's (Sun Protection Factor) and high UVA protection while being photostable.

Sun care products often exhibit a significant stickiness which results in the adherence of sand onto the skin which is highly undesirable.

It was therefore the object of the present invention to remedy the disadvantages of the prior art and to develop sun care products comprising triazine derivatives and/or amino substituted hydroxybenzophenone which exhibit a reduced stickiness resulting in a reduced affinity of sand to the skin.

Surprisingly, it has been found that compositions comprising specific benzotriazol derivatives in combination with a UV filter substance selected from the group consisting of triazine derivatives and/or an amino substituted hydroxybenzophenone exhibit a significantly reduced stickiness.

Thus, the invention relates in one aspect to topical compositions comprising at least one benzotriazol derivative of formula (I) and at least one UV filter substance selected from the group consisting of triazine derivatives and/or an amino substituted hydroxybenzophenone

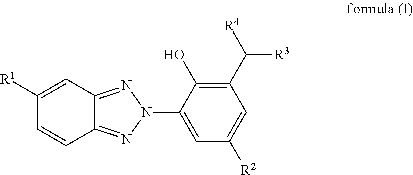

wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen.

In one advantageous embodiment, the compositions in addition contain a phosphate ester emulsifier.

In a preferred embodiment the compositions do not contain a phosphate ester emulsifier.

Another subject matter of the invention is directed to a method for reducing the sand adherence on skin after application of a topical composition comprising at least one UV filter substance selected from the group consisting of triazine derivatives and an amino substituted hydroxybenzophenone, said method comprising the step of incorporating into said composition at least one benzotriazol derivative of formula (I), with all definitions and preferences as given above. In one advantageous embodiment, the compositions furthermore comprise a phosphate ester surfactant. In another preferred embodiment the compositions do not contain a phosphate ester emulsifier.

Also the invention relates to the use of a benzotriazol derivative of formula (I) as sand repellent.

In a particular preferred embodiment the compositions according to the present invention furthermore comprise a dibenzoylmethane derivative, such as particularly butyl methoxy dibenzoylmethane.

The term “Cₓ-C_{y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethyl propyl, 1-methylpentyl, 2-methyl pentyl, 3-methylpentyl, 4-methylpentyl, 1,1-dimethyl propyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl (mono- or poly-)substituted, in particular C₁₋₅alkyl (mono- or poly-)substituted cyclic, bicyclic or tricyclic hydrocarbon residues such as in particular cyclopentyl, cyclohexyl, cycoheptyl or decahydronaphtyl. Preferably, C₅₋₁₀cycloalkyl denotes to unsubstituted or C₁₋₂alkyl (mono- or poly-)substituted cyclopentyl, cyclohexyl or cycoheptyl such as in particular to unsubstituted or (mono- or poly-)methyl substituted cyclohexyl such as most in particular cyclohexyl or 3,3,5-trimethyl-cyclohexyl.

The term “Cₓ-C_{y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

Particular suitable amino substituted hydroxybenzophenone derivatives encompass compounds of formula (II)

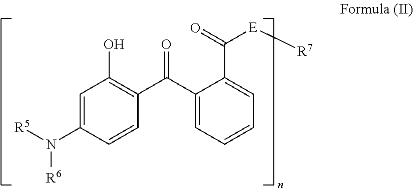

wherein
R⁵ and R⁶ independently of each other are hydrogen; C₁-C₂₀alkyl; C₂-C₂₀alkenyl; C₅-C₁₀cycloalkyl or C₅-C₁₀cycloalkenyl; or R⁵ and R⁶, together with the nitrogen atom they are bound to, form a 5 to 6 membered ring;
n is an integer from 1 or 2;

### E is —O— or —N(R⁸)— and

R⁸ is hydrogen; C₁-C₅alkyl; or C₁-C₅hydroxyalkyl;
with the proviso that
when n=1 then R⁷ is C₁-C₂₀alkyl; C₂-C₂₀alkenyl; C₁-C₅hydroxyalkyl; C₅-C₁₀cycloalkyl; C₅-C₁₀cycloalkeny; C₆₋₁₀aryl; or aralkyl optionally substituted by O, N or S; or a C₁-C₅ aminocarbonyl or alkylcarbonyl radical;
when n=2 then R⁷ is an C₁-C₂₀alkyl; C₅-C₁₀cycloalkyl-; C₂-C₂₀alkenyl- or aryl-diradical or R⁷ with E forms a diradical of formula (III) resulting in a compound of formula (IV)

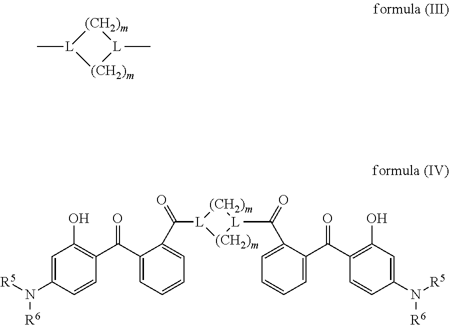

wherein L is N (nitrogen) and
m is an integer between 1 and 3.

A particular suitable amino substituted hydroxybenzophenone derivative according to the present invention is diethylamino hydroxybenzoyl hexyl benzoate [IUPAC Name: Hexyl 2-[4-(diethylamino)-2-hydroxybenzoyl]benzoate] sold under the tradename Uvinul® A plus by BASF SE Ludwigshafen.

Another suitable amino substituted hydroxybenzophenone derivative according to the present invention is 1,1′-(1,4-piperazinediyl)bis[1-[2-[4-(diethylamino)-2-hydroxybenzoyl]phenyl]-methanone [CAS No 919803-06-8] which is e.g. disclosed in WO 2007071584.

Compounds according to formula (II) and their preparation are e.g. disclosed in EP1046391, EP1133980, DE10012408 and WO 2007071584.

Particularly suitable triazine derivatives are ethylhexyl triazone (Uvinul® T-150), diethylhexyl butamido triazone (Uvasorb® HEB) and bis-ethylhexyloxyphenol methoxyphenyl triazine (Tinosorb® S) such as most preferably bis-ethylhexyloxyphenol methoxyphenyl triazine.

The amount of the at least one benzotriazol derivative of formula (I) in the compositions according to the present invention is preferable selected in the range of 1 to 20 wt.-%, such as preferably 2 to 20 wt.-%, such as more preferably in the range of 2 to 15 wt.-% and in particular in the range of 4 to 10 wt.-%, and most in particular in the range of 4 to 8 wt.-% based on the total weight of the composition.

The amount of the amino substituted hydroxybenzophenone derivative such as in particular of diethylamino hydroxybenzoyl hexyl benzoate in the compositions according to the invention is preferable selected in the range of 0.5 to 8 wt.-%, in particular in the range of 1 to 5 wt.-%, most particular in the range of 3 to 5 wt.-% based on the total weight of the composition.

The amount of the triazine derivative such as in particular of bis-ethylhexyloxyphenol methoxyphenyl triazine in the compositions according to the invention is preferable selected in the range of 0.5 to 8 wt.-%, in particular in the range of 1 to 5 wt.-%, most particular in the range of 2 to 4 wt.-% based on the total weight of the composition.

In a particular embodiment of the present invention the benzotriazol derivative is selected from compounds of formula (I) wherein R¹ and R⁴ are hydrogen, R² is methyl and R³ is C₅₋₁₀alkoxy such as preferably C₆₋₁₀alkoxy, or C₆cycloalkoxy such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124).

In another particular embodiment of the present invention the compound of formula (I) is a compound wherein R¹ and R⁴ are hydrogen, R² is methyl and R³ is undecyl (C₁₁H₂₃) which is commercially available as Tinogard TL [INCI Name: benzotriazolyl dodecyl p-cresol; IUPAC Name: 2-(2H-benzotriazol-2-yl)-6-dodecyl-4-methyl-phenol] at BASF SE Ludwigshafen.

In a further particular embodiment the benzotriazol derivative of formula (I) is benzotriazolyl dodecyl p-cresol, the triazine derivative is bis-ethylhexyloxyphenol methoxyphenyl triazine and the amino substituted hydroxybenzophenone derivative is diethylamino hydroxybenzoyl hexyl benzoate. It is particularly preferred if the topical composition furthermore comprises butyl methoxydibenzoylmethane.

In another particular embodiment the benzotriazol derivative of formula (I) is selected from compounds of formula (I) wherein R¹ and R⁴ are hydrogen, R² is methyl and R³ is C₅₋₁₀alkoxy such as in particular C₆₋₁₀alkoxy, or C₆cycloalkoxy such as most in particular 2,5,5-trimethylhexyloxy, 3,5,5-trimethylhexyloxy, isoamyloxy, 2-ethylhexyloxy or 3,3,5-trimethyl-cyclohexyloxy and the triazine derivative is bis-ethylhexyloxyphenol methoxyphenyl triazine and the amino substituted hydroxybenzophenone derivative is diethylamino hydroxybenzoyl hexyl benzoate. It is particularly preferred if the topical composition furthermore comprises butyl methoxydibenzoylmethane.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z X (x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

Particular suitable dibenzoylmethane derivatives to be used in the topical compositions according to the present invention encompass 2-methyldibenzoylmethane, 4-methyldibenzoylmethane, 4-isopropyldibenzoylmethane, 4-tert.-butyldibenzoylmethane, 2,4-dimethyldibenzoylmethane, 2,5-dimethyldi-benzoylmethane, 4,4′-diisopropyldibenzoylmethane, 4,4′-dimethoxydibenzoylmethane, 4-tert.-butyl-4′-methoxydibenzoylmethane, 2-methyl-5-isopropyl-4′-methoxydibenzoyl-methane, 2-methyl-5-tert-butyl-4′-methxydibenzoylmethane, 2,4-dimethyl-4′-methoxydi-benzoylmethane, 2,6-dimethyl-4-tert-butyl-4′-methoxydibenzoylmethane such as most in particular 4-(tert.-butyl)-4′-methoxydibenzoylmethane (INCI Name: butyl methoxy dibenzoylmethane; IUPAC Name: 1-(4-Methoxyphenyl)-3-(4-tert-butylphenyl)propane-1,3-dione) which is e.g. commercially available as PARSOL® 1789 at DSM Nutritional Products Ltd Kaiseraugst.

The amount of the dibenzoylmethane derivative such as in particular of butyl methoxydibenzoylmethane in the compositions according to the invention is preferable selected in the range of 2 to 8 wt.-%, in particular in the range of 3 to 6 wt.-%, most particular in the range of 3 to 5 wt.-% based on the total weight of the composition.

The term phosphate ester emulsifier refers to phosphate esters emulsifier of formula (II)

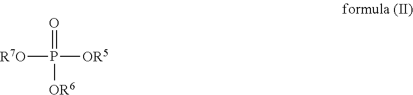

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular phosphate ester emulsifier according to the invention is potassium cetyl phosphate e.g. commercially available as Amphisol® K at DSM Nutritional Products Ltd Kaiseraugst.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations (sun care products), such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples. In a particular embodiment, the O/W emulsifier is no phosphate ester emulsifier.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W respectively Si/W emulsifier is preferably used in an amount of 0.5 to 10 wt.-% such as in particular in the range of 0.5 to 5 wt.-% such as most in particular in the range of 1 to 4 wt.-% based on the total weight of the composition.

Suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Noveon.

Another class of particularly suitable emulsifiers are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate).

Also suitable are polyalkylenglycolether such as Brij 72 (Polyoxyethylen(2)stearylether) or Brij 721 (Polyoxyethylene (21) Stearyl Ether e.g. available at Croda.

The topical compositions according to the present invention are advantageously in the form of an oil-in-water (O/W) emulsion comprising an oily phase dispersed in an aqueous phase in the presence of an O/W emulsifier selected from the group consisting of polyalkylenglycolether, polyethyleneglycol (PEG) ester or diester, non ionic self-emulsifying system derived from olive oil, as well as hydrophobically modified polyacrylic acid such as preferably from polyalkylenglycolether, polyethyleneglycol (PEG) ester or diester, non ionic self-emulsifying system derived from olive oil as well as mixtures thereof. The preparation of such O/W emulsions is well known to a person skilled in the art and illustrated in the examples.

In particular embodiment, the invention relates to topical compositions in the form of O/W emulsions comprising an oily phase dispersed in an aqueous phase in the presence of an O/W emulsifier wherein the benzotriazol derivative of formula (I) is benzotriazolyl dodecyl p-cresol, the triazine derivative is bis-ethylhexyloxyphenol methoxyphenyl triazine and the amino substituted hydroxybenzophenone derivative is diethylamino hydroxybenzoyl hexyl benzoate and the O/W emulsifier is selected from the group consisting of polyalkylenglycolether, polyethyleneglycol (PEG) esters or diesters, as well as non ionic self-emulsifying systems derived from olive oil such as particularly Arlacel 165, Olivem 1000, Brij72, and Brij 721 as well as mixtures thereof. It is particularly preferred if the topical composition furthermore comprises butyl methoxydibenzoylmethane.

In a further embodiment, the invention relates to topical compositions in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the triazine derivative is bis-ethylhexyloxyphenol methoxyphenyl triazine and the amino substituted hydroxybenzophenone derivative is diethylamino hydroxybenzoyl hexyl benzoate and the O/W emulsifier is selected from the group consisting of polyalkylenglycolether, polyethyleneglycol (PEG) esters or diesters, as well as non ionic self-emulsifying system derived from olive oil, such as particularly Arlacel 165, Olivem 1000, Brij72, and Brij 721 as well as mixtures thereof. It is particularly preferred if the topical composition furthermore comprises butyl methoxydibenzoylmethane.

In another embodiment the topical compositions according to the present invention are advantageously in the form of a water-in-oil (W/O) emulsion comprising a water phase dispersed in an oily phase in the presence of an W/O emulsifier selected from the group consisting of polyglyceryl esters/diesters. The preparation of such W/O emulsions is well known to a person skilled in the art and illustrated in the examples.

In particular embodiment, the invention relates to topical compositions in the form of water-in-oil (W/O) emulsion comprising a water phase dispersed in an oily phase in the presence of an W/O emulsifier wherein the benzotriazol derivative of formula (I) is benzotriazolyl dodecyl p-cresol, the triazine derivative is bis-ethylhexyloxyphenol methoxyphenyl triazine and the amino substituted hydroxybenzophenone derivative is diethylamino hydroxybenzoyl hexyl benzoate and the W/O emulsifier is selected from the group consisting of polyglyceryl esters or diesters such as particularly Dehymuls PGPH. It is particularly preferred if the topical composition furthermore comprises butyl methoxydibenzoylmethane.

In a further embodiment, the invention relates to topical compositions in the form of water-in-oil (W/O) emulsion comprising a water phase dispersed in an oily phase in the presence of an W/O emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the triazine derivative is bis-ethylhexyloxyphenol methoxyphenyl triazine and the amino substituted hydroxybenzophenone derivative is diethylamino hydroxybenzoyl hexyl benzoate and the W/O emulsifier is selected from the group consisting of polyglyceryl esters or diesters such as particularly Dehymuls PGPH. It is particularly preferred if the topical composition furthermore comprises butyl methoxydibenzoylmethane.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-radiation such as in particular sun-burn and/or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc. without being limited thereto.

The necessary amounts of the cosmetic and dermatological adjuvants and additives can based on the desired product easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The following examples are provided to further illustrate the compositions and effects of the present invention. These examples are illustrative only and are not intended to limit the scope of the invention in any way.

### EXAMPLE 1

### Compositions

The compositions as outlined in table 1 and 2 were prepared according to standard methods in the art.

### Sand Repellent Properties

50 mg of the respective formulation as outlined in table 2 were applied on PMMA Schonberg plates (5×5 cm). Plates are dried at 43° C. for 15 minutes. 2.5 g sand were distributed on the plates. For removal of excess sand the plates were tilt (about 45°, left and right side). The sticky sand residue on each plate was determined through weighting. The results are presented in table 2.

The reduction in the sand adherence was determined as:

ΔAdherence [%]={(Adherence Sample [mg])−(Adherence Reference [mg])}*100%.

(Samples A=Reference/Samples B=according to the invention)

TABLE 1

| | | 1A | 1B | 2A | 2B | 3A | 3B | 4A | 4B |
|---|---|---|---|---|---|---|---|---|---|
| Tradename | INCI Name | Wt.-% | | | | | | | |
| Phenonip | Preservative | 0.80 | 0.80 | 0.80 | 0.80 | 0.80 | 0.80 | 0.80 | 0.80 |
| Keltrol CG-T | Xanthan Gum | 0.30 | 0.30 | 0.30 | 0.30 | 0.30 | 0.30 | 0.30 | 0.30 |
| Finsolv TN | C12-15 Alkyl | 30.0 | 30.0 | 30.0 | 30.0 | 30.0 | 30.0 | 30.0 | 30.0 |
| | Benzoate | | | | | | | | |
| Arlacel 165 | Glyceryl Stearate, | 5.00 | 5.00 | 5.00 | 5.00 | | | 2.50 | 2.50 |
| | PEG-100 Stearate | | | | | | | | |
| Olivem 100 | | | | | | 5.00 | 5.00 | 5.00 | 5.00 |
| Lanette O | Cetearyl Alcohol | 1.50 | 1.50 | 1.50 | 1.50 | | | | |
| Tinosorb ® S | Bis-Ethylhexyl- | 4.00 | 4.00 | | | 4.00 | 4.00 | | |
| | oxyphenol Methoxy- | | | | | | | | |
| | phenyl Triazine | | | | | | | | |
| Tinogard ® TL | Benzotriazolyl | | 4.00 | | 4.00 | | 4.00 | | 4.00 |
| | dodecyl | | | | | | | | |
| | p-Cresol | | | | | | | | |
| Uvinul ® A Plus | Diethylamino Hydroxy- | | | 4.00 | 4.00 | | | 4.00 | 4.00 |
| | benzoyl Hexyl | | | | | | | | |
| | Benzoate | | | | | | | | |

| Water | Ad 100 | | | | | | | |
|---|---|---|---|---|---|---|---|---|
| Sand adherence | 1.49 | 1.41 | 1.38 | 1.36 | 1.40 | 1.38 | 1.39 | 1.38 |
| [mg] | | | | | | | | |

| | | | | |
|---|---|---|---|---|
| Δ Adherence [%] | −5% | −1.5% | −1.4% | −0.7% |

TABLE 2

| | | 5A | 5B | 6A | 6B | 7A | 7B | 8A | 8B |
|---|---|---|---|---|---|---|---|---|---|
| Tradename | INCI Name | Wt.-% | | | | | | | |
| Phenonip | Preservative | 0.80 | 0.80 | 0.80 | 0.80 | | | | |
| Keltrol CG-T | Xanthan Gum | 0.30 | 0.30 | 0.30 | 0.30 | 0.30 | 0.30 | 0.30 | 0.30 |
| Finsolv TN | C12-15 Alkyl | 30.0 | 30.0 | 30.0 | 30.0 | 30.0 | 30.0 | 30.0 | 30.0 |
| | Benzoate | | | | | | | | |
| Brij 72 | Steareth-2 | 2.5 | 2.5 | 2.5 | 2.5 | | | | |
| Brij 721 | Stearath-21 | 2.50 | 2.50 | 2.50 | 2.50 | | | | |
| Dahymuls PGPH | Polyglyceryl-2 | | | | | 6.00 | 6.00 | 6.00 | 6.00 |
| | Dipolyhydroxystearate | | | | | | | | |
| Lanette O | Cetearyl Alcohol | 1.00 | 1.00 | 1.00 | 1.00 | | | | |
| Tinosorb ® S | Bis-Ethylhexyloxy- | 4.00 | 4.00 | | | 4.00 | 4.00 | | |
| | phenol Methoxy- | | | | | | | | |
| | phenyl Triazine | | | | | | | | |
| Tinogard ® TL | Benzotriazolyl | | 4.00 | | 4.00 | | 4.00 | | 4.00 |
| | dodecyl | | | | | | | | |
| | p-Cresol | | | | | | | | |
| Uvinul ® A Plus | Diethylamino Hydroxy- | | | 4.00 | 4.00 | | | 4.00 | 4.00 |
| | benzoyl Hexyl | | | | | | | | |
| | Benzoate | | | | | | | | |
| Magnesium | | | | | | 0.50 | 0.50 | 0.50 | 0.50 |
| sulfate | | | | | | | | | |
| heptahydrate | | | | | | | | | |

| Water | Ad 100 | | | | | | | |
|---|---|---|---|---|---|---|---|---|
| Sand adherence | 1.55 | 1.48 | 1.48 | 1.46 | 1.60 | 1.55 | 1.57 | 1.49 |
| [mg] | | | | | | | | |

| | | | | |
|---|---|---|---|---|
| Δ Adherence [%] | −4.5% | −1.4% | −3.1% | −5% |

TABLE 3

| | | 9A | 9B | 10A | 10B |
|---|---|---|---|---|---|
| Tradename | INCI Name | Wt.-% | | | |
| Phenonip | Preservative | 0.80 | 0.80 | 0.80 | 0.80 |
| Keltrol CG-T | Xanthan Gum | 0.30 | 0.30 | 0.30 | 0.30 |
| Finsolv TN | C12-15 Alkyl Benzoate | 30.00 | 30.00 | 30.00 | 30.00 |
| Pemulen TR2 | Acrylates/C10-30 Alkyl Acrylate | 0.40 | 0.40 | 0.40 | 0.40 |
| | Crosspolymer | | | | |
| TEA Care | | 0.40 | 0.40 | 0.40 | 0.40 |
| Tinosorb ® S | Bis-Ethylhexyloxyphenol Methoxyphenyl | 4.00 | 4.00 | | |
| | Triazine | | | | |
| Tinogard ® TL | Benzotriazolyl dodecyl | | 4.00 | 4.00 | 4.00 |
| | p-Cresol | | | | |
| Uvinul ® A Plus | Diethylamino Hydroxy-benzoyl Hexyl | | | | 4.00 |
| | Benzoate | | | | |

| Water | Ad 100 | | | |
|---|---|---|---|---|
| Sand adherence [mg] | 1.34 | 1.32 | 1.54 | 1.41 |

| | | |
|---|---|---|
| Δ Adherence [%] | −1.5% | −8.4% |

TABLE 4

| | 9A | | 10A | |
|---|---|---|---|---|
| | (Reference) | 9B | (Reference) | 10B |
| Trade Name | Wt.-% | | | |
| Phenonip | 0.80 | 0.80 | 0.80 | 0.80 |
| Keltrol CG-T | 0.30 | 0.30 | 0.30 | 0.30 |
| Finsolv TN | 30.00 | 30.00 | 30.00 | 30.00 |
| Ampisol K | 5.00 | 5.00 | 5.00 | 5.00 |
| Lanette O | 1.50 | 1.50 | 1.50 | 1.50 |
| Tinogard TL | | 4.00 | | 4.00 |
| Tinosorb S | 4.00 | 4.00 | | |
| Uvinul A plus | | | 4.00 | 4.00 |
| Water | Ad 100 | | | |
| Sand adherence [mg] | 1.37 | 1.32 | 1.45 | 1.32 |

| | | |
|---|---|---|
| Δ Adherence [%] | −4% | −10% |

As can be retrieved from the results in tables 1 to 4, the compositions according to the present invention exhibit a reduced sand-adherence.

1. A topical composition comprising a benzotriazol derivative of formula (I) 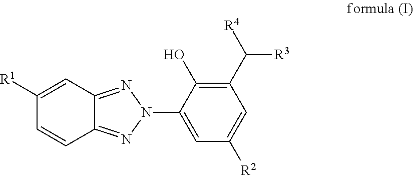
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen,
characterized in that the composition further comprises at least one UV filter substance selected from the group consisting of a triazine derivative and an amino substituted hydroxybenzophenone as well as mixtures thereof.

2. The topical composition according to claim 1, characterized in that the benzotriazol derivative is used in an amount selected in the range of 1 to 20 wt.-% based on the total weight of the composition.

3. The topical composition according to claim 1, characterized in that the benzotriazol derivative is used in an amount selected in the range of 2 to 20 wt.-% based on the total weight of the composition.

4. The topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyl or undecyl.

5. The topical composition according to claim 1, characterized in that the triazine derivative is selected from the group consisting of ethylhexyl triazone, diethylhexyl butamido triazone and bis-ethylhexyloxyphenol methoxyphenyl triazine.

6. The topical composition according to claim 1, characterized in that amino substituted hydroxybenzophenone derivative is a compound of formula (II) 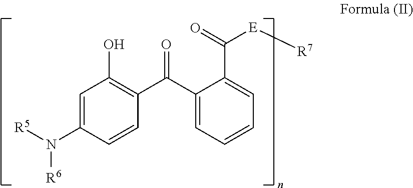
wherein
R⁵ and R⁶ independently of each other are hydrogen; C₁-C₂₀alkyl; C₂-C₂₀alkenyl; C₅-C₁₀cycloalkyl or C₅-C₁₀cycloalkenyl; or R⁵ and R⁶, together with the nitrogen atom they are bound to, form a 5 to 6 membered ring;
n is an integer from 1 or 2;
E is —O— or N(R⁸)— and
R⁸ is hydrogen; C₁-C₅alkyl; or C₁-C₅hydroxyalkyl;
with the proviso that
when n=1 then R⁷ is C₁-C₂₀alkyl; C₂-C₂₀alkenyl; C₁-C₅hydroxyalkyl; C₅-C₁₀cycloalkyl;
C₅-C₁₀cycloalkeny; C₆₋₁₀aryl; or aralkyl optionally substituted by O, N or S; or a C₁-C₅ aminocarbonyl or alkylcarbonyl radical;
when n=2 then R⁷ is an C₁-C₂₀alkyl; C₅-C₁₀cycloalkyl-; C₂-C₂₀alkenyl- or aryl-diradical or R⁷ with E forms a diradical of formula (III) 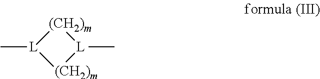
wherein L is N (nitrogen) and
m is an integer between 1 and 3.

7. The topical composition according to claim 6, characterized in that the amino substituted hydroxybenzophenone derivative is diethylamino hydroxybenzoyl hexyl benzoate or 1,1′-(1,4-piperazinediyl)bis[1-[2-[4-(diethylamino)-2-hydroxybenzoyl]phenyl]-methanone.

8. The topical composition according to claim 1, characterized in that the amount of the triazine derivative and of the amino substituted hydroxybenzophenone is selected, independently of each other, in the range of 0.5 to 8 wt.-%.

9. The topical composition according to claim 1, characterized in that the topical composition further comprises butyl methoxydi benzoyl methane.

10. The topical composition according to claim 9, characterized in that the amount of the dibenzoylmethane derivative is selected in the range of 2 to 8 wt.-% based on the total weight of the composition.

11. The topical composition according to claim 1, characterized in that the topical composition is an O/W emulsion comprising an oily phase dispersed in an aqueous phase in the presence of an O/W emulsifier.

12. The topical composition according to claim 11, characterized in that the O/W emulsifier is selected from the group consisting of polyalkylenglycolether, polyethyleneglycol (PEG) ester or diester and non ionic self-emulsifying system derived from olive oil as well as hydrophobically modified polyacrylic acid.

13. The topical composition according to claim 11, characterized in that the amount of the O/W emulsifier is selected in the range of 0.5 to 10 wt.-%, based on the total weight of the composition.

14. The topical composition according to claim 1, characterized in that the composition comprises at least one co-surfactant in an amount selected in the range of 0.1 to 10 wt.-% based on the total weight of the composition.

15. Method for reducing the sand adherence on skin after application of a topical composition comprising at least one UV filter substance selected from the group consisting of triazine derivatives and an amino substituted hydroxybenzophenone, said method comprising the step of incorporating into said composition at least one benzotriazol derivative of formula (I), 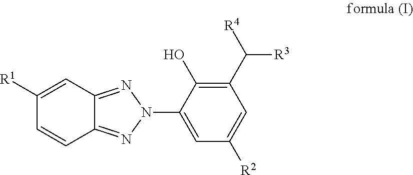
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.
